(19) United States
(12) Patent Application Publication
(10) Pub. No.: US 2008/0019956 A1
(43) Pub. Date: Jan. 24, 2008

(54) Enzymatic prevention and control of biofilm

(76) Inventor: Manoj Kumar, Palo Alto, CA (US)

Correspondence Address: JANET KAISER CASTANEDA;GENENCOR INTERNATIONAL, INC., 925 PAGE MILL ROAD, PALO ALTO, CA, 94304-1013, US

(21) Appl. No.: 11/492,294

(22) Filed: Jul. 24, 2006

(51) Int. Cl. A61K 38/54

(52) U.S. Cl. 424/94.2

(57) ABSTRACT

Described herein is a composition for removing biofilm from surfaces. The composition is an enzyme mixture having at least three enzymes and resulting in the removal of at least 40% biofilm from the surface.

### FIELD OF THE INVENTION

This invention relates to enzyme compositions and methods for preventing and removing biofilm formation upon surfaces.

### BACKGROUND OF THE INVENTION

Biofilms consist of an attached community of microorganisms embedded in a slimy exopolymer matrix that persist despite control attempts with traditional approaches designed to kill free-floating microorganisms. The resistance of biofilms to antibiotics, antiseptics, and even to oxidizing biocides has been well documented.

Despite the problems associated with unwanted biofilm growth, biofilms are useful for treating wastewater and show particular promise for recalcitrant contaminants, mixed-waste streams, and in situ bioremediation.

Enzymatic methods for biofilm prevention and/or reduction are known in the art and can be found in the following publications: WO 06/031554; WO 01/98214; WO 98/26807; WO 04/041988; W) 99/14312; and WO 01/53010.

WO 06/031554 discloses the use of an alpha-amylase derived from a bacterium for preventing, removing, reducing, or disrupting biofilm present on a surface. WO 01/98214 discloses one or more acylases and a carrier to degrade a lactone produced by microorganisms to prevent or remove biofilm. WO 98/26807 discloses the use of one or more hydrolases from a fungal source in combination with an oxidoreductase such as an oxidase, a peroxidase or a laccase to kill bacterial cells present in biofilm. WO 04/041988 discloses a detergent enzyme mixture of protease, esterase and or amylase. WO 99/14312 discloses bacterial enzyme mixtures for biofilm degradation. WO 01/53010 discloses sequential use of, first, a carbohydrase and next a protease enzyme for biofilm removal. However, the disclosures known in the literature do not efficiently address biofilm prevention and removal and have not been yet been reduced to practice.

Thus there is a need in the art for efficient products to eliminate, prevent and/or reduce biofilms in industrial, dental, and health care settings.

### BRIEF SUMMARY OF THE INVENTION

Enzymes for biofilm prevention and control are applicable for, but not limited to, industrial water management such as cooling towers, drinking water, waste water; dental hygiene, medical implants and devices, hemodialysis systems; oil recovery, bioremediation wells; paper and pulp processing; ship hulls; and food processing equipment.

In a first embodiment of the present invention, an enzyme mixture is used to prevent or reduce biofilm formation.

In a second embodiment of the present invention, greater than 40% of a biofilm is removed following treatment with an enzyme mixture.

In one aspect of the present invention, the enzyme mixture is one or more proteases, one or more glucanases, and one or more cutinases.

In another aspect of the present invention, the enzyme mixture is one or more proteases, one or more glucanases, for example a cellulase, one or more mannanases, and one or more cutinases.

In yet another aspect of the present invention, the enzyme mixture is one or more proteases, one or more glucanases, one or more mannanases, and one or more lipases. In an additional aspect of the present invention, the enzyme mixture is one or more amylases and a glucanase.

In yet another additional aspect of the present invention, the enzyme mixture is one or more amylases and one or more proteases.

In yet another additional aspect of the present invention, the enzyme mixture is one or more cellulases and one or more proteases.

The enzyme mixtures of the present invention reduce biofilm by at least about 40% by at least about 50%, by at least about 60%, by at least about 70%, by at least about 80%, by at least about 90%.

In the present invention, the enzyme mixtures are effective in preventing or reducing biofilm without the addition of a surfactant and without the use of a laccase enzyme. In the present invention, the enzyme mixtures are at least as effective as a 10% bleach treatment in removing biofilm.

The enzyme mixtures may be used to remove and prevent biofilms in industrial, dental, and health care settings. These biofilm prevention and removal applications include but not limited industrial water management such as cooling towers, drinking water, waste water, dental hygiene, medical implants and devices, hemodialysis water system, oil recovery, bioremediation wells, paper and pulp processing, ship hull, and food processing.

Other objects, features and advantages of the present invention will become apparent from the following detailed description. It should be understood, however, that the detailed description and specific examples, while indicating preferred embodiments of the invention, are given by way of illustration only, since various changes and modifications within the scope and spirit of the invention will become apparent to one skilled in the art from this detailed description.

### DETAILED DESCRIPTION

The invention will now be described in detail by way of reference only using the following definitions and examples. All patents and publications, including all sequences disclosed within such patents and publications, referred to herein are expressly incorporated by reference.

Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, N.Y. (1991) provide one of skill with a general dictionary of many of the terms used in this invention. Although any methods and materials similar or equivalent to those described herein can be used in the practice or testing of the present invention, the preferred methods and materials are described. Numeric ranges are inclusive of the numbers defining the range. Unless otherwise indicated, nucleic acids are written left to right in 5′ to 3′ orientation; amino acid sequences are written left to right in amino to carboxy orientation, respectively. Practitioners are particularly directed to Sambrook et al., 1989, and Ausubel F M et al., 1993, for definitions and terms of the art. It is to be understood that this invention is not limited to the particular methodology, protocols, and reagents described, as these may vary. Numeric ranges are inclusive of the numbers defining the range.

Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention, which can be had by reference to the specification as a whole. Accordingly, the terms defined immediately below are more fully defined by reference to the specification as a whole.

### Definitions

“Biofilm” means a community of microorganisms embedded in an extracellular polymer matrix attached to a surface. Biofilm may have one or more microorganisms and further includes water and may include other trapped particles. The microorganisms may be gram positive or gram-negative bacteria (aerobic or anaerobic); algae, protozoa, and/or yeast or filamentous fungi. In some embodiments the biofilm is living cells of bacterial genera of Staphylococcus, Streptomyces, Pseudomonas, Listeria, Streptococcus, and Escherichia.

“Surface” means any structure having sufficient mass to allow for attachment of biofilm. Hard surfaces include, but are not limited to metal, glass, ceramics, wood, minerals (rock, stone, marble, granite), aggregate materials such as concrete, plastics, composite materials, hard rubber materials, and gypsum. The hard materials may be finished with enamels and paints. Hard surfaces are found, for example in water treatment and storage equipment and tanks; dairy and food processing equipment and facilities; medical equipment and facilities, such as surgical instruments and permanent and temporary implants; industrial pharmaceutical equipment and plants. Soft surfaces are, for example, hair and all types of textiles. Porous surfaces may be biological surfaces, such as skin, keratin or internal organs. Porous surfaces also may be found in certain ceramics as well as in membranes that are used for filtration. Other surfaces include, but are not limited to, ship hulls and swimming pools.

“Enzyme dosage” means an amount of enzyme mixture, or an amount of a single enzyme used in an enzyme mixture, utilized to treat the biofilm Factors affecting enzyme dosage include, but are not limited to, the type of enzyme, the surface to be treated, and the intended result. In one embodiment, the enzyme dosage is the amount of enzyme mixture needed to reduce biofilm by at least 40%. In general, practical, economically feasible total enzyme dosage levels are about 1%. It will be understood by those skilled in the art that higher levels of enzyme may be used if desired. Equal dosages of each enzyme in the enzyme mixture may be used but are not required. In general, the enzyme content in the enzyme mixture is in total about 1% or less, 2% or less, 3% or less, 4% or less, 5% or less.

“Biofilm Removal” means at least a 40% reduction in biofilm on a surface by catalytic activity of an enzyme mixture. Removal is measured with a crystal violet assay as shown in Example 2 below wherein the assay immerses samples in a solution of crystal violet (0.31% w/v) for ten minutes prior to rinsing the samples three times in PBS to remove unbound stain. The bound stain is extracted from the biofilm using 95% ethanol and the absorbance of the crystal violet/ethanol solution is read at 540 nm. Percent removal of Pseudomonas biofilm is calculated from [(1-Fraction remaining biofilm biofilm)×100]. Fraction remaining biofilm is calculated by subtracting the absorbance of the medium+enzyme solutions from the absorbance of the solutions extracted from the enzyme treated biofilms divided by the difference in absorbance from that of untreated control biofilms minus the absorbance of the growth medium only. In other embodiments of the invention removal is at least a 50% reduction in biofilm, at least a 60% reduction in biofilm, at least a 70% reduction in biofilm, at least an 80% reduction in biofilm, at least a 90% reduction in biofilm, and at least a 100% reduction, in biofilm.

An “Enzyme Mixture” for treating biofilm means at least two enzymes. The at least two enzymes may be combinations of carbohydrases, such as cellulases, endoglucanases, cellobiohydrolases and beta-glucosidases; amylases, such as alpha amylases; proteases, such as serine proteases, eg. subtilisins; esterases and cutinases; granular starch hydrolyzing enzymes; lipases, such as phospholipase, and hemicellulases such as mannanases. The enzymes used in the enzyme mixtures may be derived from plant and animal sources, bacteria, fungi or yeast, and may be wild type or variant enzymes.

“Acid conditions”, “neutral conditions” and “basic conditions” are well known to those skilled in the art. For purposes of this disclosure, acid conditions means a pH from about 4 to 6. Neutral conditions means a pH from about 6 to 8. Basic conditions means a pH from about 8 to 10.

Hydrolases (E.C.3.) that may be used include, for example, proteases, glucanases (family 16 glycosyl hydrolase), cellulases, esterases, mannanases, and arabinases. Neutral and serine proteases, subtilisins, may be used for the present invention. Neutral proteases are proteases that have optimal proteolytic activity in the neutral pH range of approximately 6 to 8. Suitable neutral proteases are aspartate and metallo proteases. Commercially suitable metallo-proteases are MULTIFECT, PURAFECT L, FNA, PROPERASE L, PURADAX EG7000L, and GC106 from Aspergillus niger, all available from Genencor International, Inc., Palo Alto, Calif., and Alcalase, Savinase, Esperase and Neutrase (Novo Nordisk A/S, Denmark). The neutral proteases may be derived from bacterial, fungal or yeast sources, or plant or animal sources and may be wild type or variant enzymes. Variant enzymes are produced in sources that express genes that were mutated from parent genes.

Examples of cellulases that may be used for the present invention may be endoglucanases, cellobiohydrolases and beta-glucosidases, including cellulases having optimal activity in the acid to neutral pH range, for example, PURADAX derived from a bacterial source, LAMINEX and INDIAGE from Genencor International, Inc., both derived from a fungal source. Cellulases may be derived, for example, from fungi of the genera Aspergillus, Trichoderma, Humicola, Fusarium and Penicillium.

Examples of useful granular starch hydrolyzing enzymes include glucoamylases derived from strains of Humicola, Aspergillus, and Phizopus. Granular starch hydrolyzing (GSH) enzymes means enzymes that hydrolyze starch in granular form. Glucoamylase refers to the amyloglucosidase class of enzymes (e.g., EC.3.2.1.3 glucoamylase, 1,4-alpha-D-glucan glucohyrolase.). These are exo-acting enzymes which release glucosyl residues from the non-reducing ends of amylaose and amylopectin molecules. The enzyme also hydrolyzes alpha-1,6 and alpha-1,3 linkages. Glucoamylase activity may be measured using the well-known assay based on the ability of glucoamylase to catalyze the hydrolysis of p-nitrophenyl-alpha-D-glucopyranoside (PNPG) to glucose and p-mitrophenol. At an alkaline pH, the nitrophenol forms a yellow color that is proportional to glucoamylase activity and is monitored at 400 nm prior to comparison against an enzyme standard measured as a GAU. A GAU (glucoamylase activity unit) is defined as the amount of enzyme that will produce 1 gm of reducing sugar, calculated as glucose per hour from a soluble starch substrate (4% ds) at pH 4.2 and 60C. Suitable commercially available glucoamylases from Genencor International Inc. include OPTIDEX, DISTILLASE, and G-ZYME.

Examples of lipases that may be used for the present invention may be acid, neutral and alkaline lipases and phospholipases. Commercially available lipases and phospholipases from Genencor International Inc. include LYSOMAX and CUTINASE.

Examples of hemicellulase mannanases that may be used for the present invention may be GC265 from Bacillus lentus, HEMICELL and PURABRITE, bother from Bacillus lentus, from Genencor International, Inc., and the mannanases described in Stahlbrand et al, J. Biotechnol. 29 (1993), 229-242.

Examples of esterases and cutinases that may be used for the present invention may be obtained from Genencor International, Inc. from any source, including, for example bacterial sources such as Pseudomonas mendocina or fungal sources such as Humicula or Fusarium.

Examples of amylases that may be used in the present invention include alpha or beta amylases which may be obtained from bacterial or fungal sources, such as Bacillus amylases (B. amyloliquefaciens, B. licheniformis, and B. stearothermophilus) and Aspergillus, Humicola and Trichoderma amylases, for examples (A. niger, A. kawachi, and A. oryzae. Amylases may be obtained from Genencor International Inc. and include SPEZYME FRED, SPEZYME AA, CLARASE, AMYLEX and the mixture of amylases SPEZYME ETHYL. Amylases available from Novozymes A/S (Denmark) include BAN, AQUAZYM, AQUAZYM Ultra, and TERMAMYL. Other amylases are mixtures of amylases, such as M1 from Biocon, and CuConc from Sumizyme, Aris Sumizyme L (endo 1,5 alpha-L arabinase), ACH Sumizyme (beta mannase), Humicola Glucoamylase, dextranase, dextramase, chitinase, ENDOH, and Optimax L1000 (glucoamylase).

In the present invention over 375 different enzyme mixtures were tested for biofilm removal properties. The screening resulted in the identification of 33 surprising enzyme mixtures that resulted in biofilm reduction of at least 40% (69% to 84%) utilizing a high-throughput method as described in Example 1. Seventeen of the 33 enzyme mixtures were used in acid conditions and reduced biofilm by 71% to 84%, five of the enzyme mixtures were used in neutral conditions and reduced biofilm by 69% to 88%, and eleven of the enzyme mixtures were used in basic conditions.

The 33 enzyme mixtures includes mixtures of an alpha amylase and a mannanase; an amylase and a protease; an amylase and arabinase; at least one alpha amylase and at least two other amylases; a protease, cellulase and glucanase; a protease, cellulase, and three glucanases; a protease, cellulase and mannanase; a protease, cellulase and amylase; a protease, amylase, and glucanase; a protease, mannanase, and amylase; a cellulase, arabinase and amylase; a protease, cellulase, mannanase and phopholipase; a protease, glucanase, amylase, and arabinase; a protease, cellulase, and two glucanases; a protease, cellulase, and three glucanases; three proteases, a cellulase, a mannanase, and a phopholipase; three proteases, cellulase, phospholipase and esterase; three proteases, a mannanase, phospholipase and esterase; three proteases, a cellulase, and a mannanase; two proteases, cellulase, and glucanase; two proteases, cellulase, glucanase, and mannanase; two proteases, a cellulase, glucanases, phospholipase and mannanase; at least three amylases and a cellulase; an amylase, arabinase, and cellulase; an amylase, arabinase and protease; at least three amylases and a protease; at least three amylases, a protease, and a cellulase; a glucanase and an amylase mixture; a cellulase and an amylase mixture.

A preferred set of twenty enzyme mixtures includes protease, glucanase and esterase; protease glucanase, esterase and mannanase; protease, glucanase, phospholipase and mannanase; three proteases, glucanase, phospholipase and mannanase; three proteases, phospholipase, esterase and mannanase; three proteases, glucanase and mannanase; two proteases, cellulase, glucanase, phospholipase and mannanase; protease, glucanase and mannanase; protease, cellulase, phospholipase and esterase; two proteases, glucanase, phospholipase and esterase; two proteases, glucanase, phospholipase and mannanase; three proteases, cellulase, phospholipase and glucanase; three proteases, cellulase, phospholipase and mannanase; three proteases, glucanase, phospholipase and esterase; protease, cellulase, glucanase, phospholipase and esterase; two or more amylases and glucanase; at least three amylases; at least two amylases, glucanase and protease.

Four particularly preferred enzyme mixtures are: protease, glucanase and cutinase and may be prepared using the commercially availably enzymes MULTIFECT NEUTRAL; LAMINEX BG and cutinase; protease, glucanase, mannanase and cutinase, and may be prepared using the commercially availably enzymes MULTIFECT NEUTRAL; LAMINEX BG; mannanase and cutinase.; protease, glucanase, mannanase and phospholipase and may be prepared using the commercially available enzymes MULTIFECT NEUTRAL; LAMINEX BG; mannanase and LYSOMAX; and a mixture of three proteases plus cellulase, mannanase, and cutinase and may be prepared using the commercially availably enzymes PROPERASE L; PURAFECT L; FNA; LAMINEX BG, mannanase and cutinase.

Preferred embodiments of the present invention include the following commercially available enzyme preparations from Genencor International Inc.: MULTIFECT NEUTRAL; LAMINEX; LYSOMAX; PROPERASE; PURADAX, PURAFECT; and SPEZYME, all of which are registered trademarks of Genencor International, Inc.

MULTIFECT NEUTRAL comprises a Bacillus amyloliquefaciens protease (EC3.4.24.28); LAMINEX BG having an activity level or about 3200 IU/g comprises a Trichoderma B-glucanase (cellulase EC3.3.1.6); LYSOMAX having an activity level of about 400 U/g comprises a Streptomyces violceoruber phospholipase; PROPERASE having an activity level of about 1600 PU/g comprises a Bacillus alcalophilus protease (EC3.4.21.62); PURAFECT having an activity of about 42,000 GSU/g comprises a subtilisin protease (EC3.4.21.62), as described in U.S. Pat. No. 5,624,829, which is hereby incorporated by reference in its entirety; FNA comprises a Bacillus subtilis protease (EC3.4.21.62), as described in U.S. Pat. No. 34,606 and in U.S. Pat. No. 5,310,675, which are hereby incorporated by reference in its entirety; PURADAX having an activity level of about 32 U/g comprises Trichoderma reesei cellulase (EC3.2.1.4), as described in U.S. Pat. No. 5,753,484, which is hereby incorporated by reference in its entirety; SPEZYME FRED having an activity level of about 15,100 LU/g comprises an alpha amylase from Bacillus licheninformis (EC3.2.1.1), as described in U.S. Pat. Nos. 5,736,499; 5,958,739; and 5,824,532, which are hereby incorporated by reference.

Preferred enzyme mixtures using commercially available enzyme include the following:
1. MULTIFECT NEUTRAL; LAMINEX BG and cutinase.
2. MULTIFECT NEUTRAL; LAMINEX BG; mannanase and cutinase.
3. MULTIFECT NEUTRAL; LAMINEX BG; mannanase and LYSOMAX.
4. PROPERASE L; PURAFECT L; FNA; LAMINEX BG, mannanase and cutinase.
5. PROPERASE L; PURAFECT L; FNA; mannanase, cutinase and LYSOMAX.
6. PROPERATE L; PURAFECT L, FNA, mannanase, LAMINEX BG.
7. MULTIFECT NEUTRAL; LAMINEX BG; and mannanase.
8. FNA; PURADAX EG 7000L; LAMINEX BG, and cutinase.
9. PURAFECT L; FNA; LAMINEX BG; and LYSOMAX.
10. PROPERASE L; FNA; LAMINEX BG; and LYSOMAX.
11. PROPERASE L; PURAFECT L; FNA; LAMINEX BG; PURADAX EG 7000L; and LYSOMAX.
12. PROPERASE L; PURAFECT L; FNA; LAMINEX BG; cutinase and LYSOMAX.
13. MULTIFECT NEUTRAL; PURADAX EG 7000L; LAMINEX BG; LYSOMAX; and cutinase.
14. PROPERASE L; LAMINEX BG; LYSOMAX and cutinase.

More particularly preferred enzyme mixtures are the combinations 1, 2, 3 and 4 listed above. Additional particularly preferred enzyme combinations include: SPEZYME, which comprises an alpha amylase obtained from Bacillus licheniformis; CuCONC, which is the trade name for the Koji strain of Rhizopus niveus glucoamylase which has granular starch hydrolyzing activity (Shin Nihon Chemical Co. Ltd. Japan); AFP GC106, which is an acid fungal protease (Shin Hihon Chemical Co. Ltd. Japan); M1, which is available from Biocon India, Ltd., Bangalore, India); ARIS SUMIZYME (1,5-alpha arabinase), and ACH SUMIZYME.
15. SPEZYME FRED L; CuCON and LAMINEX BG.
16. SPEZYME FLRED L; Aris SUMIZYME and LAMINEX BG.
17. SPEZYME FRED L and CuCONC.
18. SPEZYME FRED L; CuCONC and GC106.
19. SPEZYME FRED L and GC106.
20. SPEZYME FRED L and M1.
21. SPEZYME FRED L; Aris SUMIZYME and GC106.
22. SPEZYME FRED L; CuCONC; LAMINEX BG and GC106.
23. SPEZYME FRED L; ACH SUMIZYME and GC106.
24. SPEZYME FRED L; Aris SUMIZYME; LAMINEX BG and GC106.
25. CuCONC and LAMINEX BG.
26. SPEZYME FRED L and Aris SUMIZYME.
27. M1 and LAMINEX BG.
28. SPEZYME FRED L; LAMINEX BG and GC106.
29. CuCONC; LAMINEX BG and GC106.

### Methodology

The enzyme mixtures of this invention are added to biofilm in amounts effective to remove biofilm. The precise dosage is not critical to the invention and may vary widely depending on the nature of the surface to be treated, and upon the treatment conditions, such as pH and temperature. In the examples, the amount of enzyme used was up to a total amount of about 1%, and in some cases from 3% to about 6%.

The method of the invention is preferably carried out within a pH range wherein the enzyme of the enzyme mixture are active. Generally the pH of the biofilm removing composition in the range of about 4 to about 9.

The method of the invention is preferably carried out at a temperature wherein the enzymes comprising the mixture are active, and generally is about 20° C. to about 50° C. In the experimental disclosure which follows, the following abbreviations apply: eq (equivalents); M (Molar); μM (micromolar); N (Normal); mol (moles); mmol (millimoles); μmol (micromoles); nmol (nanomoles); g (grams); mg (milligrams); kg (kilograms); μg (micrograms); L (liters); ml (milliliters); μl (microliters); cm (centimeters); mm (millimeters); μm (micrometers); nm (nanometers); ° C. (degrees Centigrade); h (hours); min (minutes); sec (seconds); msec (milliseconds); U (unit); IU (International Unit.

### Preparation of Enzyme Mixtures

All the various combination of enzyme mixtures for screening of efficacy against biofilm removal and prevention were prepared on a weight basis. In some instances, 1 wt % of each enzyme was mixed for a desired enzyme combination in a desired buffer, creating enzyme mixtures at a dosage of 3 wt %, 4 wt %, 5 wt %, and 6 wt %. Addition of all the enzymes was sequential and proteases were added last just prior to the start of biofilm treatment. A more economically relevant enzyme mixture for further studies was created where the combination of all enzymes in a mix was set to final total combined amount of 1%. Again, addition of all the enzymes was sequential and proteases were added last just prior to the start of biofilm treatment. The enzymes in the mixture need not be added sequentially and may be added all at the same time.

### EXAMPLES

The present invention is described in further detail in the following examples which are not in any way intended to limit the scope of the invention as claimed. The attached Figures are meant to be considered as integral parts of the specification and description of the invention. All references cited are herein specifically incorporated by reference for all that is described therein. The following examples are offered to illustrate, but not to limit the claimed invention.

A variety of enzyme mixtures were screened by testing the ability of each mixture to remove Pseudomonas aeruginosa biofilms. Screening was accomplished using a high-throughput 96-well microtiter plate method.

### Example 1

### General Experimental Setup

A high-throughput method was used for screening a large number of mixtures of enzymes based on a designed study matrix of enzymes. PBS and acetate buffers were used to prepare the solutions (Tris buffer: pH 7.0 or 8.5 and acetate buffer: pH 5.0). Various enzyme mixture combinations were made of enzymes according to pH and temperature specifications of the enzymes. Altable containing all the 375 different combinations is included as an attached Appendix A. A 96 well method was used to screen the 375 different combinations of enzymes with 4 replicates for each. This analysis allowed for the formation of biofilms in the wells of 96 well microtitre plates, which can be used to provide up to 96 different test samples. Bacterial inoculum culture (Pseudomonas aeruginosa, PO1, biofilm forming) was grown in tryptic soy broth (TSB) at 21° C. overnight in a shake flask. 20 ml of this broth was then added to another 180 ml of fresh tryptic soy broth in another shake flask. 200 ul of this diluted inoculum was then added to each well of a 96 well plate using a 96 pin replicator. At every 8-10 hours, nutrients, planktonic cells and media were aspirated and replaced with fresh TSB medium. The biofilms were grown in the wells for 24 hours at 21° C. Following biofilm formation the media was removed from the 96 well plate and the various enzyme and control treatment solutions were transferred to the wells of the plate. The biofilms were allowed to soak for a given period of time (90 minutes was used for this study). The wells were then rinsed twice, with deionized water, to remove any remaining treatment solution and suspended cells from the system. The biofilm was then stained with crystal violet for 10 minutes. The wells were each rinsed 4 times to remove any excess stain from the system, and then eluted with 300 ul of ethanol. The elution step improved the detection of stain during the analyses. The plate was then immediately read with a microtiter plate reader (J. microbiological methods 54 (2003) 269-276). All treatments were run with at least 4 replicates. Under the screening conditions, bleach controls had a 75% reduction at pH 7.0, 75% at pH 5.0, and a 93% reduction at pH 8.

### Biofilm Removal Under Acidic pH Condition

Specific enzymes such as proteases, lipases, cellulases, and other carbohydrases which are effective for their hydrolytic actions under acidic conditions (pH 5) were selected to screen for their efficacy to remove biofilm. For example, GC106 acidic protease was used for this study in combinations with lipases, cellulases and other carbohydrases which are effective in their action under acidic conditions. 17 enzyme combination matrixes out of 375 combinations screened from this study reached 69-88% biofilm removal.

### Biofilm Removal Under Neutral pH Condition

Specific enzymes such as proteases, lipases, cellulases, and carbohydrases which are effective for their hydrolytic actions under neutral conditions (pH 7) were selected to screen for their efficacy to remove biofilm. For example, a neutral protease was used for this study in combinations with lipases, cellulases and other carbohydrases which are effective in their action under neutral conditions. 5 enzyme combination matrix screened from this study reached 71-84% biofilm removal.

### Biofilm Removal Under Basic pH Condition

Specific enzymes such as alkaline proteases, lipases, cellulases, and other carbohydrases which are effective for their hydrolytic actions under alkaline conditions (pH 8.4) were selected to screen for their efficacy to remove biofilm. For example, serine proteases such as FNA, Purafact, Proparase were used for this study in combinations with lipases, cellulases and other carbohydrases which are effective in their action under basic conditions. 11 enzyme combination matrixes screened from this study reached 70-80% biofilm removal.

### Statistical Analysis of Data

The data was analyzed for statistical significance. An analysis on the variance determined the following thirty three (33) enzyme mixtures to be statistically different from the controls. The family-wise error rate was set at 0.05; that is, to be 95% confident that there will be no false positive results in one set of 143 test results from example 4. To accomplish this, set each comparison error rate to Epc=0.00036 because 0.05=1−(1−0.00036)¹⁴³. The individual results derived from this analysis are Definitely Significant, i.e., statistically significantly greater than zero at an Epc level of 0.00036. Details of this method for statistical analysis can be found at the following web site. http://core.ecu.edu/psvc/wuenschk/docs30/multcomp.doc and the concept is well illustrated at this web site. http://www.brettscaife.net/statistics/introstat/08multiple/lecture.html

Efficacious enzyme mixtures for biofilm removal of at least 40% biofilm are listed in Table 1 in their order of reduction for each of the different sets of enzymes.

TABLE 1
Biofilm removal for various enzyme combinations

| | Percent | |
|---|---|---|
| Enzyme combination | Removal | Conditions |
| Bleach Control | 93 | Basic |
| Pep 3, Cel 2, Pal 2 | 84 | Basic |
| Pep 3, Cel 2, Car 1, Pal 2 | 82 | Basic |
| Pep 3, Cel 2, Car 1, Pal 1 | 80 | Basic |
| Pep 1, 2, 4, Cel 2, Car 1, Pal 1 | 80 | Basic |
| Pep 1, 2, 4, Car 1, Pal 1, Pal 2 | 78 | Basic |
| Pep 1, 2, 4, Cel 2, Car 1 | 77 | Basic |
| Pep 2, 4, Cel 1, Cel 2, Car 1, Pal 1 | 76 | Basic |
| Pep 3, Cel 2, Car 1 | 76 | Basic |
| Pep 4, Cel 1, Pal 1, Pal 2 | 75 | Basic |
| Pep 2, 4, Cel 2, Pal 1 | 75 | Basic |
| Pep 1, 4, Cel 2, Car 1, Pal 1 | 74 | Basic |
| Bleach Control | 75 | Neutral |
| Pep 1, 2, 4, Cel 1, Cel 2, Pal 1 | 72 | Neutral |
| Pep 1, 2, 4, Cel 1, Car 1, Pal 1 | 72 | Neutral |
| Pep 1, 2, 4, Cel 2, Pal 1, Pal 2 | 72 | Neutral |
| Pep 3, Cel 1, Cel 2, Pal 1, Pal 2 | 72 | Neutral |
| Pep 1, 4, Cel 2, Pal 1, Pal 2 | 70 | Neutral |
| Bleach Control | 75 | Acid |
| Car 2&7, Cel 2 | 88 | Acid |
| Car 2&4, Cel 2 | 83 | Acid |
| Car 2&7 | 81 | Acid |
| Car 2&7, Pep 5 | 81 | Acid |
| Car 2, Pep 5 | 81 | Acid |
| Car 2&6 | 78 | Acid |
| Car 2&4, Pep5 | 78 | Acid |
| Car 2&7, Cel 2, Pep 5 | 77 | Acid |
| Car 2&5, Pep 5 | 77 | Acid |
| Car 2&4, Cel 2, Pep 5 | 77 | Acid |
| Car 7, Cel 2 | 75 | Acid |
| Car 2&4 | 75 | Acid |
| Car 6, Cel 2 | 74 | Acid |
| Car 2, Cel 2, Pep 5 | 72 | Acid |
| Car 7, Cel 2, Pep 5 | 72 | Acid |
| Car 2&6, Cel 2 | 69 | Acid |
| Car 2&5, Cel 2, Pep 5 | 69 | Acid |

### Table 2 below provides a key to identify the enzyme in the mixtures shown in Table 1.

| Code | Enzyme Name | Enzyme Type |
|---|---|---|
| CAR1 | GC265 | MANNANASE |
| CAR2 | SPEZYME FRED-L | ALPHA AMYLASE |
| CAR4 | ARIS SUMIZYME | 1,5-alpha L arabinase |
| CAR5 | ACH SUMIZYME | Beta mannanase |
| CAR6 | BIOCON M1 | Amylase mixture |
| CAR7 | CUCONO SUMIZYME | Amylase mixture |
| CEL1 | PURADAX | CELLULASE |
| CEL2 | LAMINEX BG | GLUCANASE |
| PAL1 | LYSOMAX | PHOSPHOLIPASE |
| PAL2 | CUTINASE | ESTERASE |
| PEP1 | PROPERASE | PROTEASE |
| PEP2 | PURAFECT | PROTEASE |
| PEP3 | MULTIFECT NEUTRAL | PROTEASE |
| PEP4 | FNA | PROTEASE |
| PEP5 | GC106 | PROTEASE |

Data provided by the high-throughput method was useful for screening a large number of mixtures. Further investigation of the candidate mixtures was next performed to confirm their effectiveness against biofilms, including biofilms of Pseudomonas aeruginosa, Listeria moncytogenes, Staphylococcus aureus, and drinking water consortium biofilms.

Thirty enzyme mixtures were evaluated for Pseudomonas biofilm removal and the six highest performing enzyme mixtures were used to evaluate removal of other biofilms as described below.

### Example 2

### Evaluation of Table 1 Enzyme Mixtures with DCD Biofilm Reactor

### Materials and Methods

The enzyme mixtures in Table 1 were evaluated for biofilm removal using a laboratory model system, the CDC Biofilm Reactor (model CBR 90, Biosurface Technologies Corporation, Bozeman, Mt.). This system was developed by the Centers for Disease Control and has been used to study biofilms formed by various bacterial species. The CDC Biofilm Reactor consists of a one-liter vessel with eight polypropylene coupon holders suspended from the lid. Each coupon holder can accommodate three 0.5-inch diameter sample coupons. For the experiments reported herein, the sample coupons were constructed from polystyrene, to be consistent with high-throughput screening assays that were performed using polystyrene microtiter plates. Two CDC biofilm reactors were operated in parallel providing a total of 48 sample coupons per experiment. Liquid growth medium entered through the top of the vessel and exited via a side-arm discharge port. A magnetic stir bar incorporating a mixing blade provided fluid mixing and surface shear.

### 1. Pseudomonas aeruginosa Biofilm

CDC Biofilm Reactor vessels with a working volume of approximately 400 ml containing 10%-strength tryptic soy broth medium were inoculated with P. aeruginosa and operated in batch mode (no inflowing medium) for 6 hours at 37° C. After establishing the batch culture, flow of medium at a rate of 600 ml/hr was provided for an additional 42 hours to establish P. aeruginosa biofilms on the polystyrene sample coupons. At the end of the biofilm growth period, six control coupons were removed from each of the two reactors and rinsed with sterile phosphate-buffered saline (PBS) to remove unattached bacteria. Three of the coupons from each reactor were then analyzed for biofilm using the crystal violet staining method described below. The remaining three control coupons from each reactor were sonicated in PBS, serially diluted, and plated on tryptic soy agar to enumerate the number of culturable bacteria within the biofilm.

The remaining 30 test coupons and 6 control coupons were transferred to 12-well tissue culture plates and treated with the selected high performing enzyme mixtures, used at an enzyme dosage of 1% wt total enzyme, in buffer for 90 minutes at 45° C. The six control coupons were treated with the same buffer used to prepare the enzyme mixtures. Following the treatments, the coupons were rinsed three times with PBS and analyzed for biofilm using the crystal violet staining method. This method consisted of immersing the coupons in a solution of crystal violet (0.31% w/v) for ten minutes, rinsing the coupons three times in PBS to remove unbound stain. The bound stain was then extracted from the biofilm using 95% ethanol and the absorbance of the crystal violet/ethanol solution was read at 540 nm. Percent removal of Pseudomonas biofilm was calculated from [(1-Fraction remaining biofilm biofilm)*×100]. Fraction remaining biofilm was calculated by subtracting the absorbance of the medium+enzyme solutions from the absorbance of the solutions extracted from the enzyme treated biofilms and that was divided by the difference in absorbance from that of the untreated control biofilms minus the absorbance of the growth medium only. The average thickness of the biofilms was 0.2 mm.

### 1. Pseudomonas aeruginosa Biofilm

Biofilm percent removal assessed using biofilms grown in the CDC-BR were some what lower than those determined previously using the High-Throughput Screening Assay (HTA), as shown below in Table 3. This is likely due to the more tenacious nature of biofilms grown in the CDC-BR. The CDC-BR creates a higher shear environment than the 96-well microtiter plate method used for the HTA, which likely resulted in biofilms that were more difficult to remove. Nonetheless, biofilm removal of up to 77% was observed with some of the enzyme combinations. The combination of “Pep 1,2,4, Cel 2, Car1, Pal 1” ranked ninth in the HTA tests at 80% removal but performed better than any other combination on CDC-BR biofilms with 77% removal. The highest percent biofilm removal was observed for enzyme mixtures prepared in alkaline buffer (50 mM Bis-Tris, pH 8.5).

TABLE 3
Results of biofilm removal tests using Pseudomonas aeruginosa biofilms grown
with the CDC Bioflim Reactor.

| | HTA % | CDC-BR % | CDC-BR | |
|---|---|---|---|---|
| Enzyme combination | Removal* | Removal | St. Dev. | CDC-BR n |
| Bleach Control | 75–93 | 75–93 | | |
| Pep 3, Cel 2, Pal 2 | 84 | 51 | 15 | 4 |
| Pep 3, Cel 2, Car 1, Pal 2 | 82 | 61 | 19 | 4 |
| Pep 3, Cel 2, Car 1, Pal 1 | 80 | 51 | 17 | 4 |
| Pep 1, 2, 4, Cel 2, Car 1, Pal 1 | 80 | 77 | 5 | 3 |
| Pep 1, 2, 4, Car 1, Pal 1, Pal 2 | 78 | 73 | 13 | 3 |
| Pep 1, 2, 4, Cel 2, Car 1 | 77 | 75 | 7 | 3 |
| Pep 2, 4, Cel 1, Cel 2, Car 1, Pal 1 | 76 | 58 | 13 | 3 |
| Pep 3, Cel 2, Car 1 | 76 | 51 | 26 | 3 |
| Pep 4, Cel 1, Pal 1, Pal 2 | 75 | 66 | 4 | 3 |
| Pep 2, 4, Cel 2, Pal 1 | 75 | 63 | 8 | 3 |
| Pep 1, 4, Cel 2, Car 1, Pal 1 | 74 | 59 | 23 | 3 |
| Pep 1, 2, 4, Cel 1 Cel 2, Pal 1 | 72 | 42 | 21 | 3 |
| Pep 1, 2, 4, Cel 1, Car 1, Pal 1 | 72 | 74 | 9 | 3 |
| Pep 1, 2, 4, Cel 2, Pal 1, Pal 2 | 72 | 58 | 28 | 3 |
| Pep 3, Cel 1, Cel 2, Pal 1, Pal 2 | 71 | 60 | 9 | 4 |
| Car 2&7, Cel 2 | 88 | 66 | 6 | 3 |
| Car 2&4, Cel 2 | 83 | 67 | 5 | 3 |
| Car 2&7 | 81 | 51 | 11 | 2 |
| Car 2&7, Pep 5 | 81 | 37 | 25 | 3 |
| Car 2, Pep 5 | 81 | 35 | 32 | 2 |
| Car 2&6 | 78 | 15 | 20 | 2 |
| Car 2&4, Pep 5 | 78 | 28 | 4 | 2 |
| Car 2&7, Cel 2, Pep 5 | 77 | 35 | n/a | 1 |
| Car 2&5, Pep 5 | 77 | 31 | n/a | 1 |
| Car 2&4, Cel 2, Pep 5 | 77 | 24 | n/a | 1 |
| Car 7, Cel 2 | 75 | 36 | 33 | 3 |
| Car 2&4 | 75 | 50 | 12 | 3 |
| Car 6, Cel 2 | 74 | 26 | 4 | 2 |
| Car 2, Cel 2, Pep 5 | 72 | 9 | 7 | 2 |
| Car 7, Cel 2, Pep 5 | 72 | 54 | 18 | 3 |

Testing of 30 enzyme mixtures using the CDC Biofilm Reactor system with Pseudomonas aeruginosa revealed twenty enzyme mixtures with biofilm removal greater than 40%, 19 mixtures with biofilm removal percentages greater than 50%, 10 mixtures with biofilm removal percentages greater than 60% and 4 mixtures above 70%. Most of the preferred enzyme mixtures found to be most effective for Pseudomonas biofilm removal are in alkaline to neutral conditions based on both HTP and CDC Reactor based biofilm removal analysis of Pseudomonas aeruginosa biofilm. Under acidic conditions, the highest performing mixture found was Car2+Car7+Cel2. Cel2 was found in most of the efficacious enzyme mixtures tested for Pseudomonas biofilm removal.

### Example 3

The following enzyme mixtures were tested to see their efficacy against three other major commercially relevant biofilm based upon CDC data on Pseudomonas and a separate HTP study on a dental biofilm, four species model. In considering the practical time available for cleaning and the possible economical dosage. The cleaning enzyme mixture contact time with biofilm coupons was reduced to 40 minutes and final enzyme concentration of all the enzyme components in the enzyme mixtures was limited to a total of 1%. For example, the PEP5+CAR2+CEL3 enzyme mix contained 0.33+0.33+0.34% of each enzyme making the final enzyme mixture dosage for testing at 1%.

Testing was conducted at the three pH's listed below using six enzyme mixtures listed below. The tests were conducted on Listeria, staphylococcus, and drinking water iofilms.

pH 5.5

1. PEP5+CAR2+CEL3

2. PEP5+CAR2+CEL2

pH 7.0

1. PEP6+PAL2+CEL3

2. PEP3+PAL2+CEL2

pH 8.5

1. PEP1+PEP2+PEP4+CEL2+CAR1+PAL1

2. PEP4+CEL1+PAL1+PAL2

### Example 4

### Listeria moncytogenes Biofilm

CDC Biofilm Reactor vessels having stainless steel coupons with a working volume of approximately 400 ml containing 10%-strength Brain Heart Infusion (B3HI) medium were inoculated with 4 ml of an overnight culture of Listeria monocytogenes (ATCC 19112) in 10% Brain Heart Infusion (BHI) at 37° C. The CRD reactor was operated in a batch mode for 24 hours followed by the continuous feed of flowing (BHI medium) at 7 mls/min for the next 24 hours. After 48 hours (24 batch+24 continuous), the reactor was stopped and dismantled. Sterile tweezers were used to remove all the stainless steel coupons from the wands, touching the front and back of the coupons as little as possible, and the coupons were placed in sterile 12 well plates for treatment. A total of 24 coupons per reactor were available and three coupons each were treated with each enzyme mixture combination (six combinations, total 1% enzyme mix concentration of all the enzyme components combined, 40 minutes, 45° C. Three coupons were not treated and were used as untreated controls. All of the treated coupons were removed from treatment and rinsed in PBS three times and placed in 75% crystal violet solution (Protocol Crystal Violet) in a twelve well plate, for ten minutes. After staining, the coupons were rinsed in PBS three times and placed in 5.0 ml 95% ethanol and placed on the shaker at room temperature for 5 minutes to elute the crystal violet. The eluted solutions were then pipetted into cuvettes and read on the spectrophotometer at 540 nm. Percent removal of Listeria biofilm was calculated from [(1-Fraction remaining biofilm biofilm)*100]. Fraction remaining biofilm was calculated by subtracting the absorbance of the medium+enzyme solutions from the absorbance of the solutions extracted from the enzyme treated biofilms and that was divided by the difference in absorbance from that of the untreated control biofilms minus the absorbance of the growth medium only.

TABLE 4
Results of Listeria biofilm removal by Genencor
enzyme mixtures using CDC reactor

| | CDC-BR % | CDC-BR St. | |
|---|---|---|---|
| Enzyme combination | Removal | Dev. | CDC-BR n |
| Bleach Control (Basic) | 93 | | |
| Bleach Control (Neutral) | 75 | | |
| Bleach Control (Acid) | 75 | | |
| PEP5 + CAR2 + CEL3 | 39 | 8 | 3 |
| PEP5 + CAR2 + CEL2 | 30 | 35 | 3 |
| PEP3 + PAL2 + CEL2 | 40 | 2 | 3 |
| PEP6 + PAL2 + CEL3 | 41 | 10 | 3 |
| PEP4 + CEL1 + PAL1 + | 51 | 21 | 3 |
| PAL2 | | | |
| PEP1 + PEP2 + PEP4 + | 56 | 13 | 3 |
| CEL2 + CAR1 + PAL1 | | | |

All the six enzyme mixtures tested showed some efficacy towards removal of Listeria biofilm, and four alkaline pH based enzyme combinations provided more than 40% biofilm removal, particularly the enzyme mixture PEP1+PEP2+PEP4+CEL2+CAR1+PAL1.

### Example 5

### Staphylococcus aureus Biofilm

CDC Biofilm Reactor vessels having polyurethane coupons with a working volume of approximately 400 ml containing 10% Tryptic soy broth medium (TSB) were inoculated with 4 ml of an overnight culture of Staphylococcus aureus (SRWC-10943) in 10% TSB medium at 37° C. The CDC reactor was operated in a batch mode for 24 hours followed by the continuous feed of flowing (TSB medium) at 7 mls/min for the next 24 hours. After 48 hours (24 batch+24 continuous), the reactor was stopped and dismantled. Sterile tweezers were used to remove all the polyurethane coupons from the wands, touching the front and back of the coupons as little as possible, and the coupons were placed in sterile 12 well plates for treatment. A total of 24 coupons per reactor were available and three coupons each were treated with each enzyme mixture combination (seven combinations, a total of 1% enzyme mix concentration of all the components combined, 40 minutes, 45° C.). Three coupons were not treated and were used as untreated controls. All of the treated coupons were removed from treatment and rinsed in PBS three times and placed in 75% crystal violet solution (Protocol Crystal Violet) in a twelve well plate, for ten minutes. After staining, the coupons were rinsed in PBS three times and placed in 5.0 ml 95% ethanol and placed on the shaker at room temperature for 5 minutes elute the crystal violet. The eluted solutions are then pipetted into cuvettes and read on the spectrophotometer at 540 nm. Percent removal of Listeria biofilm was calculated from [(1-Fraction remaining biofilm biofilm)*100]. Fraction remaining biofilm was calculated by subtracting the absorbance of the medium+enzyme solutions from the absorbance of the solutions extracted from the enzyme treated biofilms and that was divided by the difference in absorbance from that of the untreated control biofilms minus the absorbance of the growth medium only.

TABLE 5
Results of Staphylococcus aureus biofilm removal
by enzyme mixtures using CDC Reactor

| | CDC-BR % | CDC-BR St. | CDC-BR |
|---|---|---|---|
| Enzyme combination | Removal | Dev. | n |
| Bleach control | 93, 75, 75 | | |
| (Basic, Neutral, Acid) | | | |
| PEP5 + CAR2 + CEL3 | 25 | 8 | 3 |
| PEP5 + CAR2 + CEL2 | 30 | 10 | 3 |
| PEP3 + PAL2 + CEL2 | 36 | 8 | 3 |
| PEP6 + PAL2 + CEL3 | 32 | 19 | 3 |
| PEP4 + CEL1 + PAL1 + PAL2 | 28 | 18 | 3 |
| PEP1 + PEP2 + PEP4 + | 41 | 8 | 3 |
| CEL2 + CAR1 + PAL1 | | | |

All the six enzymes tested showed some efficacy towards removal of Listeria biofilm, and one alkaline pH based enzyme combination provided at least 40% biofilm removal.

### Example 6

### Drinking Water Consortia Biofilm

A 20 L sterile carboy with 19 L of BAC/GAC drinking water (containing low CFU mixed drinking water consortium) and 1 L of carbon amendment solution of the following ingredients was prepared to achieve the additional carbon concentration.

| | |
|---|---|
| L-glutamic acid | 0.0047 g/L |
| L-aspartic acid | 0.0053 g/L |
| L-serine | 0.0055 g/L |
| L-alanine | 0.0047 g/L |
| D+ glucose | 0.0048 g/L |
| D+ galactose | 0.0048 g/L |
| D− arabinose | 0.0048 g/L |

Sterile CDC reactors were placed in a laminar flow hood and were filled to the outlet with BAC/GAC water. In the 37° C. incubator, connection of all the tubing to the inlet and outlet was made and CDC reactors were placed on the stir plate. The reactors were run for 24 hours in batch followed by the continuous flow of carbon supplemented BAC/GAC water) at 7 mls/min for the next 24 hours. At the end of 48 hours (24 batch+24 continuous), the influent flow was turned off, the media was poured out from the reactors into a waste container and the reactors were placed in a laminar flow hood.

Using sterile tweezers all the coupons were removed from the wands, without touching the front and back of the coupons. PVC coupons containing drinking water consortia biofilm were then placed in sterile 12 well plates for treatment.

A total of 24 coupons per reactor were available and three coupons each were treated with each enzyme mixture combination (seven combinations, a total of 1% concentration, 40 minutes, 45° C.). Three coupons were not treated and were used as untreated controls. All of the treated coupons were removed from treatment and rinsed in PBS three times and placed in 75% crystal violet solution (Protocol Crystal Violet) in a twelve well plate, for ten minutes. After staining, the coupons were rinsed in PBS three times and placed in 5.0 ml 95% ethanol and placed on the shaker at room temperature for 5 minutes elute the crystal violet. The eluted solutions were then pipetted into cuvettes and read on the spectrophotometer at 540 nm. Percent removal of Listeria biofilm was calculated from [(1-Fraction remaining biofilm biofilm)*100]. Fraction remaining biofilm was calculated by subtracting the absorbance of the medium+enzyme solutions from the absorbance of the solutions extracted from the enzyme treated biofilms and that was divided by the difference in absorbance from that of the untreated control biofilms minus the absorbance of the growth medium only.

TABLE 7
Results of Drinking water consortia Biofilm removal
by Genencor enzyme mixtures using CDC Reactor

| | CDC-BR % | CDC-BR St. | CDC-BR |
|---|---|---|---|
| Enzyme combination | Removal | Dev. | n |
| Bleach control | 93, 75, 75 | | |
| (Basic, Neutral, Acid) | | | |
| PEP5 + CAR2 + CEL3 | 7 | 29 | 4 |
| PEP5 + CAR2 + CEL2 | 12 | 18 | 4 |
| PEP3 + PAL2 + CEL2 | 47 | 22 | 4 |
| PEP6 + PAL2 + CEL3 | 43 | 16 | 4 |
| PEP4 + CEL1 + PAL1 + PAL2 | 53 | 8 | 4 |
| PEP1 + PEP2 + PEP4 + | 59 | 8 | 4 |
| CEL2 + CAR1 + PAL1 | | | |

All the six enzymes tested showed some efficacy towards removal of drinking water consortia biofilm, and four alkaline pH based enzyme combinations provided more than 40% biofilm removal, particularly the enzyme mixture PEP1+PEP2+PEP4+CEL2+CAR1+PAL1.

The most efficacious enzyme mixture for all types of biofilm removal was a combination of FNA, Purafect L, Properase L, Laminex BG, Mannanase GC265, and Lysomax under mild alkaline conditions. For neutral to acidic pH conditions, although not as effective as those mentioned above, the enzyme mixture was comprised of Multifect Neutral, Laminex BG, and Cutinase enzymes.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A composition for removing biofilm from a surface, the composition comprising an enzyme mixture having at least two different enzymes selected from protease, cellulase, esterase, mannanase, glucanase, phospholipase and amylase.

2. The composition of claim 1 wherein the enzyme mixture is selected from protease, glucanase and esterase; protease glucanase, esterase and mannanase; protease, glucanase, phospholipase and mannanase; three proteases, glucanase, phospholipase and mannanase; three proteases, phospholipase, esterase and mannanase; three proteases, glucanase and mannanase; two proteases, cellulase, glucanase, phospholipase and mannanase; protease, glucanase and mannanase ; protease, cellulase, phospholipase and esterase; two proteases, glucanase, phospholipase and esterase; two proteases, glucanase, phospholipase and mannanase; three proteases, cellulase, phospholipase and glucanase; three proteases, cellulase, phospholipase and mannanase; three proteases, glucanase, phospholipase and esterase; protease, cellulase, glucanase, phospholipase and esterase; two or more amylases and glucanase; at least three amylases; at least two amylases, glucanase and protease.

3. The composition of claim 1 wherein the enzyme mixture is selected from protease glucanase, esterase and mannanase; three proteases, glucanase, phospholipase and mannanase; three proteases, phospholipase, esterase and mannanase; three proteases, glucanase and mannanase; protease, cellulase, phospholipase and esterase; two proteases, glucanase, phospholipase and esterase; three proteases, cellulase, phospholipase and mannanase; protease, cellulase, glucanase, phospholipase and esterase; two or more amylases and glucanase; and at least three amylases.

4. The composition of claim 1 wherein the enzyme mixture is selected from three proteases, glucanase, phospholipase and mannanase; three proteases, phospholipase, esterase and mannanase; three proteases, glucanase and mannanase; and three proteases, cellulase, phospholipase and mannanase.

5. A composition for removing biofilm from a surface comprising an enzyme mixture, the enzyme mixture consisting of three proteases, glucanase, phospholipase and mannanase.

6. The composition of claim 5 wherein the proteases are from Bacillus subtilis EC 3.3.2.6 and Bacillus alcalophilus EC 3.4.2.6, the glucanase is from Trichoderma species EC 3.3.1.6, the phospholipase is from Streptomyces species EC 3.1.1.4, and the mannanase is from Bacillus lentus.

7. The composition of claim 1 wherein said protease is selected from basic, neutral, or acidic proteases.

8. The composition of claim 1 wherein the protease is selected from the following commercially available proteases: PROPERASE, PURAFECT, MULTIFECT NEUTRAL, FNA AND GC106.

9. The composition of claim 1 wherein the cellulase is commercially available PURADAX.

10. The composition of claim 1 wherein the esterase is commercially available CUTINASE.

11. The composition of claim 1 wherein the mannanase is commercially available GC265 or HEMICELL.

12. The composition of claim 1 wherein the glucanase is commercially available LAMINEX BG.

13. A composition for removing biofilm at a neutral or basic pH consisting essentially of a mixture of enzymes selected from the group consisting of protease, glucanase and esterase; protease glucanase, esterase and mannanase; protease, glucanase, phospholipase and mannanase; three proteases, glucanase, phospholipase and mannanase; three proteases, phospholipase, esterase and mannanase; three proteases, glucanase and mannanase; two proteases, cellulase, glucanase, phospholipase and mannanase; protease, glucanase and mannanase; protease, cellulase, phospholipase and esterase; two proteases, glucanase, phospholipase and esterase; two proteases, glucanase, phospholipase and mannanase; three proteases, cellulase, phospholipase and glucanase; three proteases, cellulase, phospholipase and mannanase; three proteases, glucanase, phospholipase and esterase; protease, cellulase, glucanase, phospholipase and esterase; two or more amylases and glucanase; at least three amylases; at least two amylases, glucanase and protease.

17. The composition of claim 16 further including an endo-arabinase.

18. A composition for cleaning biofilms at acidic pH's comprising a mixture consisting essentially of a mixture of enzymes selected from the group consisting of a mixture of amylases and glucanase; amylase, arabinase and glucanase; amylase and arabinase; and an amylase mixture, glucanase and protease.

19. A method for reducing biofilm on a surface comprising:
a) providing an enzyme mixture selected from protease, glucanase and esterase; protease glucanase, esterase and mannanase; protease, glucanase, phospholipase and mannanase; three proteases, glucanase, phospholipase and mannanase; three proteases, phospholipase, esterase and mannanase; three proteases, glucanase and mannanase; two proteases, cellulase, glucanase, phospholipase and mannanase; protease, glucanase and mannanase; protease, cellulase, phospholipase and esterase; two proteases, glucanase, phospholipase and esterase; two proteases, glucanase, phospholipase and mannanase; three proteases, cellulase, phospholipase and glucanase; three proteases, cellulase, phospholipase and mannanase; three proteases, glucanase, phospholipase and esterase; protease, cellulase, glucanase, phospholipase and esterase; two or more amylases and glucanase; at least three amylases; at least two amylases, glucanase and protease;
b) applying the mixture to biofilm on a surface for a time sufficient to reduce biofilm by at least 40%.

20. The method of claim 19 wherein a) further comprises providing an enzyme mixture having about 1% to about 6% enzyme.

22. The method of claim 19 wherein the biofilm is Pseudomonas aeruginosa, Listeria monocytogenes, or Staphylococcus aureus.
